(19) United States
(12) Patent Application Publication
(10) Pub. No.: US 2020/0038421 A1
(43) Pub. Date: Feb. 6, 2020

(54) CANNABINOID AND SUGAR ALCOHOL COMPLEX, METHODS TO MAKE AND USE

(71) Applicant: Axim Biotechnologies, Inc., New York, NY (US)

(72) Inventors: George Anastassov, New York, NY (US); Lekhram Changoer, Ridderkerk (NL)

(21) Appl. No.: 16/653,416

(22) Filed: Oct. 15, 2019

Related U.S. Application Data
(63) Continuation of application No. 15/012,045, filed on Feb. 1, 2016, now abandoned.
(60) Provisional application No. 62/111,013, filed on Feb. 2, 2015.

(51) Int. Cl. A61K 31/704; A61K 31/7034; A61K 9/68; A23G 4/12; A61K 31/05; A61K 31/352; A23G 1/40; A61K 31/047; A21D 2/18; A23G 3/36; A21D 2/36; A61K 9/14; A23G 1/32; A23G 4/10; A23G 3/42; A23G 4/06

(52) U.S. Cl. CPC A61K 31/704; A23G 4/06; A61K 9/0058; A23G 4/12; A61K 31/05; A61K 31/352; A23G 1/40; A61K 31/047; A21D 2/181; A23G 3/36; A23G 3/364; A21D 2/36; A61K 9/145; A23G 1/32; A23G 4/10; A23G 3/42; A61K 31/7034

(57) ABSTRACT

The present invention generally relates to a sugar alcohol and cannabinoid complex, and methods to prepare this complex from cannabinoid oil comprising at least one cannabinoid. The complex is in solid form and may be used in food, pharmaceutical, cosmetic formulations, and medical devices wherein solid forms of cannabinoid are desirable. This complex also enhances release of active cannabinoids in oral consumption. Methods to make this complex are also disclosed.

### CROSS-REFERENCE TO RELATED APPLICATIONS

This application is a continuation of U.S. patent application Ser. No. 15/012,045, filed on Feb. 01, 2016, which claims the benefit of U.S. Provisional Application No. 62/111,013 filed Feb. 02, 2015. Each of the above-referenced patent applications is incorporated by reference in its entirety.

### BACKGROUND OF THE INVENTION

### Field of the Invention

This invention generally relates to a complex, which solidifies oily material matrix into powder matrix while increasing water solubility of the oily material. Methods to form this complex are also disclosed. This complex may enable incorporation of the oily material into various food, cosmetic, and medical device products, especially where powder form of the oily material is preferred.

### Description of the Related Technology

The cannabis plant has many naturally occurring substances that are of great interest in the fields of science and medicine. Isolated compounds from the cannabis plant include Δ⁹-tetrahydrocannabinol (THC), cannabidiol (CBD), cannabichromene (CBC), cannabigerol (CBG), cannabinol (CBN), cannabidivarin (CBDV), among other compounds. While THC has psychoactive effects, CBD, CBC, CBG, and CBDV do not. Isolated compounds from the cannabis plant are called cannabinoids. There are a total of eighty-five (85) cannabinoids that have been isolated from the cannabis plant. Many researchers have confirmed the medicinal value of cannabinoids. Cannabinoids have been investigated for possible treatment of seizures, nausea, vomiting, lack of appetite, pain, arthritis, inflammation, and other conditions.

The IUPAC nomenclature of THC is (-)-(6aR,10aR)-6,6,9-trimethyl-3-pentyl-6a, 7,8,10a-tetrahydro-6H-benzo[c]chromen-1-ol. CBD's IUPAC nomenclature is 2-((1S,6S)-3-methyl-6-(prop-1-en-2-yl) cyclo-hex-2-enyl)-5-pentylbenzene-1,3-diol). CBC has the IUPAC nomenclature of 2-methyl-2-(4-methylpent-3-enyl)-7pentyl-5-chromenol. These are among the most prominent compounds in the family of compounds extracted from the cannabis plant referred to as cannabinoids.

Cannabinoids can be isolated by extraction or cold pressing from cannabis plants. Plants in the cannabis genus include Cannabis sativa, Cannabis ruderalis, and Cannabis indica. These plants are the natural sources of cannabinoids. Cannabinoids are also available in synthetic forms. Methods to synthesize cannabinoids in lab settings were discovered and are still currently practiced. Synthetic cannabinoids are more targeted, in that the synthetic compound usually comes isolated without other cannabinoids mixed in.

Nabilone (racemic(6aR, 10aR)-1-hydroxy-6,6-dimethyl-3-(2-methyloctan-2-yl)-7,8,10, 10a-tetrahydro-6H-benzo[c]chromen-9(6aH)-one), a synthetic cannabinoid, is believed to have fewer undesired side effects than THC. Nabilone mimics the chemical compound structure of THC. THC also exists in synthetic form under the name Dronabinol ((-)-(6aR,10aR)-6,6,9-trimythel-3-pentyl-6a, 7,8,10a-tetrahydro-6H-benzo[c]chromen-1-ol)). The U.S. Food and Drug Administration approved nabilone for treatment of chemotherapy-induced nausea and vomiting. In the United States, nabilone is marketed under the name Cesamet®.

Cannabidiol (CBD) has the IUPAC name of 2-[(1R,6R)-6-isopropenyl-3-methylcyclohex -2-en-1-yl]-5-pentylbenzene-1,3-diol. CBD is non-psychoactive and is shown to have anti-psychotic effects in clinical studies on schizophrenia patients. Selected strains of marijuana and hemp, both of the species Cannabis sativa L., have been bred to produce elevated levels of CBD, up to 16% CBD in the plant material. CBD is also studied as a possible therapeutic agent for many physical and mental indications.

Cannabigerol (CBG) has an IUPAC name of 2-[2E)-3,7-dimethylocta-2,6-dienyl]-5 -pentyl-benzene-1,3-diol. CBG is also a non-psychoactive cannabinoid, and is more common in hemp than marijuana plants. CBG may be obtained as a natural constituent of cannabis or hemp extract.

Cannabinol (CBN) is a weak psychoactive cannabinoid found in Cannabis sativa and Cannabis indicia. CBN is present at lower concentration in these two cannabis species. CBN's IUPAC name is 6,6,9-trimethyl-3-methyl-benzochromen-1-ol.

A sugar alcohol is a kind of alcohol prepared from sugar. They are white, water-soluble solids that occur naturally and are used widely in the food industry as thickeners and sweeteners. In commercial foodstuffs, they are commonly used in place of sucrose (table sugar), often in combination with a high intensity artificial sweetener to counter the low sweetness. Unlike table sugar, sugar alcohols do not cause the formation of tooth cavities. Sugar alcohols occur naturally and today are often obtained by hydrogenation of sugars. While alcohol sugars do not cause cavities, they do affect blood sugar levels, albeit less than sucrose. Sugar alcohols are popular alternatives to sucrose because they contain one-third to one-half less calories than sucrose. Sugar alcohols, including those discussed below, are labeled GRAS (generally recognized as safe).

Isomalt is one type of sugar alcohol, used primarily for its sugar-like physical properties. Its energy value is only 2 kcal/gram, which is half that of sucrose. Isomalt does not promote dental caries, and is thus preferred in oral formulations. Isomalt is an equimolar mixture of two disaccharides, glucose and mannitol, and glucose and sorbitol.

Mannitol is another type of alcohol sugar that looks and tastes like sucrose. It has several medical benefits, including use in osmotherapy to treat head injuries. In fact, it is on the World Health Organization's List of Essential Medicines. A group of researchers in Israel have done studies that possibly suggest treatment for Parkinson's disease by using mannitol. Mannitol is also used as a sweetener in food and when completely dissolved in a product, produces a strong cooling effect.

Sorbitol is a sugar alcohol with a sweet taste which the human body metabolizes slowly. Most sorbitol comes from corn syrup, but it can also be found in other fruits. It is a sugar substitute that has approximately 60% of the sweetness of sucrose. It provides dietary energy at 2.6 kcal/g. It can be found in diet foods, diet sodas, sugar-free chewing gum, cough syrup, and mints. Sorbitol can also be used in cosmetics as a humectant and thickener and is often used in mouthwash and toothpaste.

Xylitol is another popular sugar alcohol that is used as a sweetener. It is roughly as sweet as sucrose with 33% fewer calories. It helps reduce dental cavities and is helpful to tooth remineralization. It contains 2.4 kcal/g as opposed to sucrose, which contains nearly 4 kcal/g. It is considered safe for diabetics and individuals with hyperglycemia. Xylitol has no known toxicity in humans.

Cannabinoids produced from natural sources usually come in oily forms. Cannabinoids are typically hydrophobic. When combined in pharmaceutical or food products, hydrophobicity and oily characteristics of cannabinoids pose certain problems to formulation. For example, cannabinoid oil when incorporated into a chewing gum matrix may face challenges in release rate due to its oily nature. Lozenge formulations also prefer solid cannabinoids.

### SUMMARY

This invention relates to a complex of at least one cannabinoid and at least one sugar alcohol, wherein the ratio of cannabinoid:sugar alcohol may be at 1:5 to 1:30. The complex may be produced by dissolution of cannabinoid and sugar alcohol in a solvent; and the solvent may be evaporated under reduced pressure. Alternatively, the complex may be produced by mixing a sugar alcohol with a cannabinoid and homogenization of the resulting solid mix. Co-precipitation of cannabinoid and at least one sugar alcohol in an organic solvent followed by freeze drying may also produce this complex. The complex is in solid form and may be incorporated into food, pharmaceuticals, cosmetic products, and medical devices.

### ABBREVIATIONS

CBC: Cannabichromene

CBD: Cannabidiol

CBDV: Cannabidivarin

CBG: Cannabigerol

CBN: Cannabinol

IUPAC: International Union of Pure and Applied Chemistry

THC: Tetrahydrocannabinol

### DETAILED DESCRIPTION OF CERTAIN INVENTIVE EMBODIMENTS

This present invention is capable of being embodied in various forms. The description below of several embodiments is made with the understanding that the present disclosure is to be considered as an exemplification of the claimed subject matter, and is not intended to limit the attached claims to the specific embodiments illustrated. The headings used throughout this disclosure are provided for convenience only and are not to be construed to limit the claims in any way. Embodiments illustrated under any heading may be combined with embodiments illustrated under any other heading.

As used herein, the verb “to comprise” in this description, claims, and other conjugations are used in their non-limiting sense to mean those items following the word are included, but items not specifically mentioned are not excluded.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more.”

The word “cannabinoid” used in this description, claims, and other conjugations is used to mean any compound that interacts with a cannabinoid receptor and other cannabinoid mimetics, including, but not limited to, certain tetrahydropyran analogs (Δ⁹-tetrahydrocannabinol, Δ⁸-tetrahydrocannabinol,6,6,9-trimythel-3-pentyl-6H-dibenzo[b,d]pyran-1-ol,3-(1,1-dimethylheptyl)-6, 6a7,8,10,10a-hexahydro-1-1hydroxy-6,6-dimythel-9H-dibezo[b,d]pyran-9-ol, (-)-(3S,4S)-7-hydroxy-delta-6-tetrahydrocannabinol-1,1-dimethylheptyl,(+)-(3S,4S)-7-hydroxy -Δ-6-tetrahydrocannabinol, and Δ⁸-tetrahydrocannabinol-1 1-oic acid); certain piperidine analogs (e.g., (-)-(6S,6aR,9R,10aR)-5,6,6a,7,8,9,10,10a-octahydro-6-methyl-1-3-[(R)-1-methyl-4-phenylbutoxy]-1,9-phenanthridinediol 1-acetate)); certain aminoalkylindole analogs (e.g., (R)-(+) -[2,3-dihydro-5-methyl-3-(4-morpholinylm-ethyl)-pyrrolo[1,2,3,-de]-1,4-benzoxazin-6-yl]-1-naphthelenyl-methanone); certain open pyran-ring analogs (e.g., 2-[3-methyl-6-(1-methylethenyl-2-cyclohexen -1-yl]-5-pentyl-1, 3 -benzendi-ol, and 4-(1,1-dimethylheptyl)-2,3′-dihydroxy-6-α-(3-hydroxypropyl) -1′,-2′,3′,4′,5′,6′-hexahydrobiphenyl), their salts, solvates, metabolites, and metabolic precursors.

The word “cannabidiol” refers to cannabidiol and cannabidiol derivatives. As used in this application, cannabidiol is obtained from industrial hemp extract with a trace amount of THC or from cannabis extract using high-CBD cannabis cultivars.

The word “cannabigerol” refers to cannabigerol and cannabigerol derivatives. As used in this application, cannabigerol is industrial hemp extract with a trace amount of THC or from cannabis extract.

The word “cannabinol” refers to cannabinol and cannabinol derivatives. As used in this application, cannabinol is obtained from cannabis extract or from industrial hemp extract.

Cannabinoids derived from natural sources usually come in oily form, known as cannabis oil, hashish oil, or hemp oil, depending on its origin. Cannabis sativa L.'s seed oil with naturally occurring cannabinoid content may also be used. The oil has a liquid to paste-like profile at room temperature, and is hydrophobic. In certain situations, cannabinoids in oily form are unsuitable for incorporation into food, cosmetics, pharmaceutical preparations, or medical devices. In these cases, cannabinoids in powder form may be preferred.

In these embodiments, cannabinoid oil has naturally occurring cannabinoids, and may contain THC, CBD, CBG, CBN, and other cannabinoids. Cannabinoid oil is derived from naturally source as an extraction from Cannabis sativa plants and/or seeds. Cannabinoid oil high in THC should be used where THC is desired. Similarly, cannabinoid oil high in CBD, CBG, or CBN should be used where CBD, CBG, or CBN is desired. Cannabinoid oil may have at least one cannabinoid present.

Sugar alcohols such as isomalt, mannitol, maltitol, lactitol, xylitol, erythritol, and sorbitol are widely used in food, cosmetics, and pharmaceutical preparations. Apart from a mildly sweet taste, sugar alcohols also dissolve readily in water.

The cannabinoid-sugar alcohol complex may be in powder form with a cannabinoid:sugar alcohol ratio at 1:5 to 1:30, preferably at 1:5 to 1:10. This complex may have at least one cannabinoid and at least one sugar alcohol. More than one cannabinoid and more than one sugar alcohol may be present.

The cannabinoid-sugar alcohol complex may be formed in suspension phase using an organic solvent to facilitate co-precipitation. Effective organic solvents may be ethanol or isopropyl alcohol. Ethanol may be used as the solvent due to its low density and suitability for human consumption as food. Isopropyl alcohol may evaporate quickly, such that solvent residue in the harvested complex may be at a minimal amount. Alternatively, the cannabinoid-sugar alcohol complex may be formed in solid phase using kneading or slurry methods.

In an embodiment, cannabinoid-sugar alcohol complex may be prepared using the co-precipitation method. A selected cannabinoid and at least one sugar alcohol may be dissolved in an organic solvent. The ratio of cannabinoid to sugar alcohol may be at 1:5 to 1:30. The amount of the organic solvent used may be 3-20 times the total weight of cannabinoid and sugar alcohol to be precipitated. The amount of solvent use may be adjusted higher to facilitate dissolution prior to co-precipitation.

Suitable organic solvents for co-precipitation may be ethanol or isopropyl alcohol. Suitable sugar alcohol may be isomalt, mannitol, sorbitol, xylitol, lactitol, maltitol, or erythritol. Cannabinoids used in these embodiments may be in powder form, and may be Δ⁹-tetrahydrocannabinol, cannabidiol, cannabinol, or cannabigerol.

The temperature at which the co-precipitation may be carried out may be room temperature, but slightly higher temperature, around 5-10° C. higher than room temperature, may be suitable for co-precipitation. After dissolution in the solvent by mixing, the solution may be set aside for 1-3 days to allow equilibrium to be reached. The solution may be freeze dried to obtain a powder complex containing the cannabinoid and sugar alcohol.

In embodiments, cannabinoid-sugar alcohol complex may be prepared using the slurry method. In this embodiment, cannabinoid oil containing at least one cannabinoid may be added into a solvent and stirred, then a sugar alcohol may be added into the same slurry. The slurry may be stirred for at least 15 minutes to form a uniform mixture. Thereafter, the slurry may be subject to heat application while under concurrent vacuum application to evaporate the solvent. The evaporated solvent may be collected in a cold trap immersed in liquid nitrogen. After the solvent is evaporated, the remaining solid may be harvested, with an off-white to green-yellow color.

The sugar alcohol to be used in these embodiments may be isomalt, mannitol, maltitol, lactitol, xylitol, erythritol, or sorbitol. Generally, the weight ratio of cannabinoid to sugar alcohol may be at 1:5 to 1:30, preferably at 1:5 to 1:10.

The solvent added to this slurry may be at 1.4 to 3 times the weight of the sugar alcohol used. The solvent may facilitate the mixing of cannabinoid and sugar alcohol. When ethanol is used in this embodiment, ethanol is of food grade, as ethanol residue may be left in the complex thereafter. When isopropyl alcohol is used, it may be completely evaporated, since isopropyl alcohol is low in density.

During evaporation of the solvent, the slurry may be under reduced pressure preferably produced by a vacuum pump. The pressure in the container may be at 100 mmHg to 300 mmHg. Higher pressure may slow the evaporation process, but pressure at up to 500 mmHg may be used. Evaporated solvent may be captured in a glass trap immersed in liquid nitrogen.

Heat application during solvent evaporation may be by means of a heat plate or a heater jacket wrapped around a container. The heater's temperature may be set at between 45° C. to 60° C. Temperature control means to change and control temperature may be used to control the heating means.

Solvent residue in the cannabinoid-sugar alcohol complex may be present. Solvent residue may be present at 0.1 to 10 by weight percent of the original added amount. Preferably, solvent may be evaporated from the complex such that solvent is reduced to 0.1 to 2 by weight percent of the original added amount. Isopropyl alcohol may be evaporated completely due to its low density.

In others embodiment, the cannabinoid-sugar alcohol complex may be formed by kneading cannabinoid oil with a sugar alcohol. The mixture may be homogenized by mixing, by pressure, or by introduction of a gas stream into the solid mix. After homogenization, the complex formed may be harvested. The weight ratio of cannabinoid to sugar alcohol in this complex may be at 1:5 to 1:30.

The cannabinoid-sugar alcohol complex may be formulated into different pharmaceutical, food, medical devices, and cosmetic compositions. This complex may be particularly useful where the oily form of cannabinoid is undesirable.

In food compositions, this cannabinoid-sugar alcohol complex may be incorporated into lozenges, chewing gums, chewable candies, hard candies, cakes, chocolate bars, granola bars, nut bars, other confectionary preparations, and drink preparations. The food composition may comprise other food components, such as starch, sugar, sugar alcohols, nuts, eggs, milk, chocolate powder, cream, water, emulsifiers, food preservatives, and other ingredients common in food.

In chewing gum formulations, oils may bind with the gum matrix, impeding the release of active components in the oil. The cannabinoid-sugar alcohol complex may increase the release rate in such chewing gum formulations.

In chewing gum formulations, the cannabinoid-sugar alcohol complex may be incorporated in a separate granule inside the chewing gum body. The granule may comprise, in addition to the cannabinoid-sugar alcohol complex, selected enhancers or bulking materials. The granule may be in any shape but contained within the chewing gum body. Multiple granules contained within the chewing gum body may also be used.

The cannabinoid-sugar alcohol complex may be incorporated in a pharmaceutical composition suitable for sublingual, buccal, dermal, oral, or rectal administration. The pharmaceutical composition may contain, in addition to the complex as described herein at pharmaceutically acceptable amounts, pharmaceutically acceptable carriers, adjuvants, or vehicles. The pharmaceutical composition may be in tablet, capsule, pill, lozenge, patch, dissolvable strip, spray mist, suppository, or pastille form.

The cannabinoid-sugar alcohol complex may be incorporated in a cosmetic preparation suitable for dermal application. The cosmetic preparation may comprise a cosmetically acceptable bulking agent, carrier, or filler. The cosmetic preparation may be in cream, lotion, liquid, ointment, balm, tablet, powder, gel, stick, or aerosol form.

In medical devices, the cannabinoid-sugar alcohol complex may be incorporated or administered for desirable properties of cannabinoid. Nonwoven wound dressing fabric, super absorbers, gauze, or surgical pads may be embedded with this complex to utilize antiseptic properties of the cannabinoids. The nonwoven fabric or super absorbers embedded with this complex may also be used for feminine hygiene products, such as sanitary napkins, pads, or tampons.

### EXAMPLES

### Example 1

In this example, a powder containing THC-isomalt complex is prepared.

Add 2 grams of Δ⁹-THC oil at 90% THC by weight into 40 mL of ethanol (95% purity, food grade) and stir. The resulting slurry is added into 20 grams of isomalt then stirred for at least 15 minutes. The slurry is placed in a flask, and the flask is placed on a heat plate. Set the heat plate to 50° C. and apply continuous vacuum to the flask by a top connector connected to a vacuum pump with a glass trap immersed in liquid nitrogen. Pressure is reduced to between 100 mmHg to 300 mmHg. Ethanol is evaporated under reduced pressure condition. Continue to apply reduced pressure and heat until the slurry becomes a solid. Harvest the solid and grind if needed.

### Example 2

In this example, a powder containing CBD-isomalt complex is prepared.

Add 1 gram of CBD oil at 90% CBD by weight into 20 mL of isopropyl alcohol (99% purity) and stir. The resulting slurry is added into 10 grams of isomalt and stirred for 20 minutes. The slurry is placed in a flask, which is set on a heat plate. Set the heat plate at 50° C. and apply vacuum to the flask. Connect the vacuum line to a glass trap immersed in liquid nitrogen to capture evaporated isopropyl alcohol. Pressure in the flask is reduced to between 100 mmHg to 300 mmHg during the evaporation. Continue to apply heat and vacuum pressure until the slurry in the flask becomes a solid. Harvest the solid and grind if needed.

### Example 3

In this example, a CBD-isomalt complex powder is used in a multi-layer chewing gum to enhance release rate.

0.12 grams of CBD-isomalt complex according to Example 2 are obtained. The CBD-isomalt complex and a bulking agent form a separate granule in the chewing gum, such that CBD does not bind with the gum matrix. CBD is released during chewing of the gum.

All references, including publications, patent applications, and patents cited herein are hereby incorporated by reference to the same extent as if each reference were individually and specifically indicated to be incorporated by reference and were set forth in its entirety herein.

It will be readily apparent to those skilled in the art that a number of modifications and changes may be made without departing from the spirit and the scope of the present invention. It is to be understood that any ranges, ratios, and range of ratios that can be derived from any of the data disclosed herein represent further embodiments of the present disclosure and are included as part of the disclosure as though they were explicitly set forth. This includes ranges that can be formed that do or do not include a finite upper and/or lower boundary. Accordingly, a person of ordinary skill in the art will appreciate that such values are unambiguously derivative from the data presented herein.

What is claimed is:

1. A method to prepare a cannabinoid and sugar or cannabinoid and sugar alcohol complex comprising the steps of:
adding at least one cannabinoid into a solvent and stir into a mixture;
adding sugar alcohol or mixture of sugar alcohols into the cannabinoid-solvent mixture above to form a cannabinoid and sugar alcohol slurry;
placing the cannabinoid and sugar alcohol slurry in a container;
stirring the cannabinoid and sugar alcohol slurry for at least 15 minutes to form a uniform mixture;
applying heat to the container to raise temperature while applying vacuum to reduce the internal pressure in the container;
collecting evaporated solvent in a cold trap;
stopping the heat and vacuum application; and
harvesting the cannabinoid and sugar alcohol solid mixture.

2. The method of claim 1, wherein the solvent is selected from the group consisting of isopropyl alcohol and ethanol.

3. The method of claim 1, wherein the sugar alcohol is selected from the group consisting of isomalt, mannitol, sorbitol, xylitol, lactitol, maltitol, and erythritol.

4. The method of claim 1, wherein the at least one cannabinoid is selected from the group consisting of Δ⁹-tetrahydrocannabinol, cannabidiol, cannabinol, and cannabigerol.

5. The method of claim 1, wherein the temperature is between 45° C. to 60° C.

6. The method of claim 1, wherein the internal pressure in the container is between 100 mmHg to 300 mmHg.

7. The method of claim 1, wherein the at least one cannabinoid is provided as cannabis oil, hashish oil, or hemp oil.

8. The method of claim 1, wherein the at least one cannabinoid is provided in powder form.

9. The method of claim 1, wherein the solvent is at 1.4 to 3 times the weight of the sugar, sugar alcohol, mixture of sugar, or mixture of sugar alcohols.

10. The method of claim 1, wherein the weight ratio based on dry weight of cannabinoid to sugar alcohol in the solid mixture is at 1:5 to 1:30.

11. The method of claim 1, wherein heat is applied to the container by means of a heat jacket or a hot plate.
